(19) United States
(12) Patent Application Publication
(10) Pub. No.: US 2020/0222348 A1
(43) Pub. Date: Jul. 16, 2020

(54) BIMODAL MODIFIED RELEASE COMPOSITIONS FOR CANCER TREATMENT

(71) Applicant: MUSLI THYROPEUTICS LTD.

(72) Inventors: Osnat ASHUR-FABIAN, Zur-Moshe (IL); Aleck HERCBERGS, Beachwood, OH (US); Yoram SELA, Raanana (IL)

(21) Appl. No.: 16/611,858

(22) PCT Filed: May 1, 2017
(86) PCT No.: PCT/IL2018/050478

§ 371 Date: Dec. 23, 2019

(51) Int. Cl. A61K 31/198; A61K 31/513; A61K 31/4164; A61K 9/00

(52) U.S. Cl. CPC A61K 31/198; A61K 31/513; A61K 9/0019; A61K 9/0053; A61K 31/4164

(57) ABSTRACT

The present invention discloses bimodal modified release fixed-dose combination (FDC) compositions and therapeutic methods for treatment of cancer by administration to a patient in need thereof of a FDC composition comprising a first active agent which is triiodothyronine (T3) or a T3 functional analog formulated to be delivered in the pulsatile delivery mode. and a second active agent which is at least one anti-thyroid agent, formulated to be delivered in immediate release (IR) mode, sustained release (SR) mode or combined immediate/slow release (IR/SR) mode.

### CROSS REFERENCE TO RELATED APPLICATIONS

This application claims priority to U.S. Provisional Patent Application Ser. No. 62/503,039 filed on May 8, 2017, the entire content of which is hereby incorporated by reference in its entirety.

### TECHNICAL FIELD

The present invention relates to bimodal modified release compositions and therapeutic methods for their use in cancer treatment.

### BACKGROUND ART

Ashur-Fabian O. and Hercbergs A. disclosed in U.S. Provisional Patent Application No. 61/328,825, filed on Apr. 28, 2010 a therapeutic method which employs treatment of a disease such as cancer with triiodothyronine (triiodothyroxine or T3) together with an anti-thyroid treatment such as treatment with an anti-thyroid agent, treatment with radioiodine and surgical removal of all or part of the thyroid gland. A pharmaceutical composition comprising T3 and an anti-thyroid agent and a kit comprising the same were also encompassed by the invention. Both the PCT Patent Application WO11135574 and the U.S. Patent Application No. US2013129615 claim a pharmaceutical composition comprising triiodothyronine (T3) or biochemical analog thereof and at least one anti-thyroid agent. The anti-thyroid agent may be a thioamide based drug which may be propylthiouracil (PTU) or Methimazole.

According to the above patent documents, the T3 or biochemical analog thereof and the anti-thyroid agent may be provided to the subject in need thereof separately, or in a single composition. However, no details were provided in the above patent documents on the nature of the composition or the amounts or dosage of the individual active agents in the single composition.

### DISCLOSURE OF INVENTION

This invention provides novel bimodal modified release fixed-dose compositions (FDC) and therapeutic methods for their use for cancer treatment.

The compositions of the present invention comprise
1. A first active agent which is triiodothyronine (T3), or functional analog thereof and
2. A second active agent which is an anti-thyroid agent.

The anti-thyroid agent may be at least one thioamide based drug.

The thioamide drug may be propylthiouracil (PTU) or methimazole or combinations thereof.

Fixed-dose combinations (also named FDC, fixed-dose compositions or fixed dose combination drugs) include two or more active pharmaceutical ingredients (APIs, henceforth referred to as active agents or actives) in a single dosage form in fixed-doses. The FDCs have a number of advantages over the regular combination therapy including the administration of two or more drugs in separate dosage forms. The most important advantage of the FDC treatments is improved patient compliance (Roger Collier, “Reducing the “pill burden” [1], CMAJ Feb. 7, 2012 vol. 184).

The FDC compositions of this invention include a first active agent which is triiodothyronine (T3) or functional analog thereof and a second active agent which is at least one anti-thyroid agent, wherein the two active agents are delivered in bimodal delivery manner (each active agent is delivered in its own delivery mode), with a delivery profile suited to the therapeutic needs of cancer treatment.

Thus, in the composition of this invention, triiodothyronine (T3) or a functional analog thereof is formulated to be delivered in pulsatile delivery mode of 2-5 pulses, preferably 2-3 pulses per 24 hrs, mimicking the administration of 2-5, preferably 2-3 daily doses of a composition comprising only the T3 or functional analog. The at least one anti-thyroid agent, on the other hand, is formulated to be delivered in immediate (IR) release mode or slow release (SR) mode or combined immediate/slow release (IR/SR) mode mimicking the administration of a single IR or IR/SR dose of a composition comprising only this active.

There is an unmet need for fixed-dose combination compositions comprising triiodothyronine (T3) or functional analog thereof and at least one anti-thyroid agent having a delivery profile suited to the therapeutic needs of cancer treatment.

### BEST MODES FOR CARRYING OUT THE INVENTION

This invention provides novel modified release fixed-dose compositions (FDC) and therapeutic methods for their use for cancer treatment.

Fixed-dose combinations (also named FDC, fixed-dose compositions or combination drugs) include two or more active pharmaceutical ingredients (APIs, henceforth referred to as active agents or actives) in a single dosage form in fixed doses. FDCs have a number of advantages over the regular combination therapy including the administration of two or more drugs in separate dosage forms. The most important advantage of the FDC treatments is improved patient compliance (Roger Collier, “Reducing the “pill burden” [1], CMAJ Feb. 7, 2012 vol. 184).

On the other hand, the strengths of the individual active agents in the FDC must be carefully chosen, to address the therapeutic needs and optimally treat the disease.

A disadvantage of the FDC is the limitation of the clinicians to prescribe the most appropriate strengths to the specific patient. This disadvantage is addressed in this invention by making available the FDC combination products in several alternative strengths.

Another aspect of the treatment of various diseases with FDCs including two or more drugs, wherein each drug has its own optimal regimen of administration. Thus, for example, one of the actives in the FDC may need to be administered in immediate release mode and the second active needs to be released in sustained release mode or several times daily.

The present invention addresses this problem by formulating the compositions of this invention in a bimodal modified release mode.

In some embodiments, the compositions of the present invention comprise
1. A first active agent which is triiodothyronine (T3), or functional analog thereof and
2. A second active agent which is at least one anti-thyroid agent.

The anti-thyroid agent may be at least one thioamide based drug.

The thioamide drug may be propylthiouracil (PTU) or methimazole or combinations thereof.

As used herein, the term “functional analog” of T3 denotes a compound exhibiting similar biochemical and pharmacological properties to T3 such as any compound having a thyronine backbone and exhibits at least partial T3 hormonal activity. In some embodiments, the T3 functional analog is an L-isomer of T3. In some other embodiments, the T3 functional analog is an iodine derivative of thyronine. In yet some other embodiments, the T3 functional analog is an L-isomer of T3—liothyronine sodium.

Preferably, the T3 functional analog is an iodine derivative of thyronine. Such iodinated derivatives may be prepared by methods known in the art, such as that described by Gunther Hillman in Great Britain patent No. GB 851,610 titled: Methods of producing iodine derivatives of thyronine. A non-limiting list of iodine derivatives of thyronine include 3′,5′,3-triiodothyronine (rT3), rT3 derived hormone, such as 3′,3-diiodothyronine, 3′,5′-diiodothyronine, 5′,3-diiodothyronine, 3′-iodothyronine, 5′-iodothyronine, 3iodothyronine, 3′-bromo-5′-iodo-3,5-di-iodothyronine, 3′-chloro-5′-iodo-3,5-diiodothyronine, isomer of 3,5,3′-triiodothyronine, e.g. triiodothyropropionic acid as well as the L-isomer of tri-iodothyronine. In one particular embodiment of the invention, the functional analog is liothyronine sodium, being the L-isomer of triiodothyronine (LT3), (marketed under the brand name Cytomel™ or Tertroxin™ in Australia).

Other T3 functional analogs may include amides of iodothyronine compounds, such as those described by Cree Gavin et al. in U.S. Pat. No. 4,426,453 titled “Derivatives of iodothyronine compounds and their use in an assay for the free iodothyronine compounds”.

The functional analog of T3 should exhibit at least partial T3 hormonal activity sufficient to, for example, signal the hypophysis that there is enough T3 in the circulation and resulting in reduction of T4 production. T4 is thyroxine (3,5,3′,5′-tetraiodothyronine, T4), which is the major hormone secreted by the follicular cells of the thyroid gland. Triiodothyronine (T3) is a thyroid hormone similar to T4 but with one fewer iodine atoms per molecule. Thyroid-stimulating hormone (TSH), secreted from the hypophisis, controls the production of T4 and T3. In the thyroid, T4 is converted to T3, however, most of the circulating T3 is formed peripherally by deiodination of T4 (85%). Thus, T4 acts as prohormone for T3.

The term “anti-thyroid treatment” is used to denote treatment with an anti-thyroid agent, treatment with radioiodine (1¹³¹) to destroy the thyroid gland or thyroidectomy treatment to remove part or all of the thyroid gland (thyroidectomy treatment may consist of hemithyroidectomy (or “unilateral lobectomy”) in which only half of the thyroid is removed or isthmectomy in which the band of tissue (or isthmus) connecting the two lobes of the thyroid is removed).

The anti-thyroid agent, as defined herein, is any chemical or biological agent that is capable of decreasing the amount of thyroid hormone produced by the thyroid gland. There are a variety of anti-thyroid agents known in the art.

In some embodiments, the anti-thyroid agent is at least one thioamide based drug.

Without being limited thereto, the anti-thyroid agent is selected from propylthiouracil (PTU), methimazole (also known as Tapazole™ or Thiamazole™) and Carbimazole.

### Bimodal Modified Release Compositions for Cancer Treatment

The modified bimodal release FDC compositions of this invention provide the tools to treat a cancer patient in need thereof with a fixed-dose combination drug including a first active agent which is triiodothyronine (T3) or functional analog thereof and a second active agent which is at least one anti-thyroid agent, wherein each active agent is delivered in its own delivery mode (bimodal delivery), suited to the therapeutic needs of cancer treatment.

Thus, triiodothyronine (T3) or a functional analog thereof are formulated to be delivered in pulsatile mode, mimicking the administration of a few immediate release doses of a composition comprising only this active.

The at least one anti-thyroid agent, on the other hand, is formulated to be delivered in an immediate release mode or slow release mode or combined IR/SR release mode comprising only the second active, the anti-thyroid agent.

The pulse delivery of T3 or functional analog is advantageous for a number of reasons.

Thyroid suppression is an effective way to reduce T4 levels; however, consequently it lowers T3 levels as well, resulting in a clinically hypothyroid state. In addition, it is well known that thyroid hormones exert negative feedback control over the hypothalamus as well as anterior pituitary, thus controlling the release of both TRH (thyrotropin-releasing hormone) from hypothalamus and TSH (thyroid-stimulating hormone) from anterior pituitary gland and thus T4 secretion from the thyroid. Interestingly, reports of addition of T3 to a patient having a normally functioning thyroid gland (euthyroid patient) show a reduction of T4 levels, arguably through activation of the hypothalamic-pituitary-thyroid (HPT) axis. Titration by T3 in accordance with the patient's clinical status is easily facilitated given the rapid absorption of T3 in addition to its short biological half-life (about 48-60 hours). The Tₘₐₓ in serum is about 4 hrs. When T3 is adequately titrated, two main benefits occur:

1. Symptoms of classical clinical hypothyroidism are obviated.

2. T3 itself is effective at inhibition of both TRH release from hypothalamus and TSH release from anterior pituitary gland in physiological doses in the (euthyroid patient) and as such, further inhibits the production and release of T4 into the blood stream.

Based on the time needed to obtain the maximal level in serum and the T3's short half-life time, the best results are achieved when administering T3 or the T3 functional analog at 6-8 hour intervals. It appears that the pituitary gland will sense these small pulses of T3 and thus will react in reducing the TSH levels, hence the FT4 (free T4) levels more quickly. Having this mechanism in a special formulation which allows for the drug to be taken once or at most twice a day is advantageous to the treatment of cancer patients.

In some embodiments, there is provided a bimodal modified release fixed-dose combination (FDC) composition comprising a first active agent which is triiodothyronine (T3) or a functional analog thereof and a second active agent which is at least one anti-thyroid agent.

In some embodiments, the bimodal modified release FDC composition of this invention comprises a T3 functional analog selected from an iodine derivative of thyronine, 3′,5′,3-triiodothyronine (rT3), rT3 derived hormone, 3′,3-diiodothyronine, 3′,5′-diiodothyronine, 5′,3-diiodothyronine, 3′-iodothyronine, 5′-iodothyronine, 3-iodothyronine, 3′-bromo-5′-iodo-3,5-di-iodothyronine, 3t-chloro-5′-iodo-3,5-diiodothyronine, isomer of 3,5,3′-triiodothyronine, triiodothyropropionic acid, L-isomer of tri-iodothyronine or an amide of iodothyronine.

In some embodiments, the bimodal modified release FDC composition of this invention comprises at least one anti-thyroid agent which is at least one thioamide based drug.

In some other embodiments, the bimodal modified release FDC composition of this invention comprises at least one thioamide based drug which is propylthiouracil (PTU), Methimazole, Carbimazole or combinations thereof.

In some embodiments, the triiodothyronine (T3), or T3 functional analog thereof of the bimodal modified release FDC composition is delivered in pulsatile release mode and the at least one anti-thyroid agent is delivered in immediate release (IR), sustained-release (SR) or combined IR/SR delivery modes.

In some embodiments, the bimodal modified release FDC composition of this invention comprises the amounts of triiodothyronine (T3) or T3 functional analog thereof and of the at least one anti-thyroid agent necessary for once daily administration to a cancer patient in need thereof.

In some embodiments, the bimodal modified release FDC composition of this invention is delivered in bimodal mode, wherein the T3 or T3 functional analog is delivered in pulsatile delivery mode, in 2-6 pulses per 24 hrs, preferably 2-3 pulses per 24 hrs.

In some other embodiments, in the bimodal modified release FDC composition of this invention the at least one anti-thyroid agent is delivered in immediate-release delivery mode.

In some other embodiments, in the bimodal modified release FDC composition of this invention the at least one anti-thyroid agent is delivered in sustained-release delivery mode, wherein at least 80% of the anti-thyroid agent is delivered over a 24 hrs period.

In some embodiments, in the bimodal modified release FDC composition of this invention, the at least one anti-thyroid agent is delivered in a combined immediate release/sustained-release delivery mode, and wherein at least 80% of the anti-thyroid agent is delivered over a 24 hrs period. In some embodiments, the bimodal modified release FDC composition of this invention, is formulated as a tablet or a capsule for oral administration.

In some other embodiments, the bimodal modified release FDC composition of this invention is formulated as an injectable composition for depot administration.

In some embodiments, the modified release FDC composition of this invention, comprises from 15 μg to 90 μg triiodothyronine (T3) or a T3 functional analog thereof and from 10 mg to 1200 mg of at least one anti-thyroid agent.

In some other embodiments, the modified release FDC composition of this invention comprises from 5 μg to 40 μg triiodothyronine (T3) or a T3 functional analog thereof and from 10 mg to 80 mg of methimazole, or from 100 mg to 1200 mg propylthiouracil (PTU) for daily administration.

In some embodiments, the modified release FDC composition of this invention comprises from 10 μg to 100 μg triiodothyronine (T3) or a T3 functional analog thereof and from 5 mg to 100 mg of methimazole, or from 20 mg to 1000 mg propylthiouracil (PTU) for daily administration.

In some embodiments, there is provided a method of treatment of cancer by administration of the bimodal modified release FDC composition of this invention to a patient in need thereof.

In some other embodiments, in the above method of treatment of cancer the cancer is solid cancer.

In some other embodiments, in the above method of treatment of cancer the solid cancer is selected from the group consisting of vascularized tumors, gonadal cancers, gastrointestinal, esophageal cancer, Barrett's esophagus cancer, oral cancer, parotid cancer, nasopharyngeal cancer, thyroid cancer, CNS cancers, urogenital cancers, skin cancers bone and connective tissue cancers.

In some embodiments, there is provided a method of treatment of solid cancer, wherein the solid cancer is glioma.

In some other embodiments, there is provided a method of treatment of glioma solid cancer, wherein the glioma is glioblastoma.

In some embodiments, there is provided a method of treatment of metastases from solid cancers, such as brain metastases.

In some embodiments, the method of treatment of cancer by administration of the FDC composition of this invention is accompanied by anti-thyroid treatment with radioiodine and/or surgical removal of all or part of the thyroid gland.

In some embodiments, there is provided a regimen of administration of the bimodal modified release FDC composition of this invention by administration to a cancer patient in need thereof of 1-4 tablets or capsules per day.

In some other embodiments, there is provided a regimen of administration of the bimodal modified release FDC composition of this invention by administration to a cancer patient in need thereof of one tablet or capsule of the FDC composition per day.

### INDUSTRIAL APPLICABILITY

The present invention provides modified release FDC compositions for the treatment of cancer comprising two active agents, a first active agent which is triiodothyronine (T3) or functional analog thereof and a second active agent which is at least one anti-thyroid agent, wherein each active agent is delivered in its own delivery mode (bimodal delivery), suited to the therapeutic needs of cancer treatment.

### EXAMPLES

The following examples illustrate certain embodiments of the invention but are not meant to limit the scope of the claims in any way. They should be regarded as exemplary embodiments. The following examples are put forth so as to provide those of ordinary skill in the art with a complete disclosure and description of how to make and use the described invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Bimodal Modified Release FDC Tablets or Bead-Filed Capsules—Methimazole in IR Mode and T3 in Pulsatile Delivery Mode

### Preparation of T3 Beads

Weigh 100 gr sugar beads (non-pareils or NP), average size 500 microns

Solubilize in 50:50 ethanol/water mixture about 2 gr Liothyronine, together with 0.1 gr PVP K30 and 0.02 gr NaCl.

Spray the T3/PVP solution over the NP's, using a Fluidized bed apparatus equipped with Wurster column.

Weigh the IR NP-coated T3 and split into three subgroups:

### 1. Non-Coated, IR Beads

### 2. Prepare a Coating Solution Comprising of Ethylcellulose 7 cp 80%, Triethylcitrate (TEC) 10% and PEG 6000 10%.

2. Extended (Delayed) Release (ER) Beads with Different Release Times for Pulsatile Delivery
2a—Coat the Second IR T3 Coated Subgroup with 10% w/w of the Previous Composition, Enabling Delayed Release of the T3 after 8 Hrs Lag Time.
2b—Coat the Third IR T3 Coated Subgroup with 25% w/w of the Previous Composition, Enabling Delayed Release of the T3 after 18 Hrs Lag Time.

Weigh 100 gr sugar beads (non-pareils), average size of 500 microns

3. Solubilize in 50:50 Ethanol/Water Mixture about 20 gr Methimazole and 1 gr of PVP

### K30

### Methimazole Beads

4. Spray the Methimazole/PVP Solution Over the NP's, Use a Fluidized Bed Apparatus Equipped with Wurster Column, Creating Methimazole IR Beads.
5. Mix the Methimazole IR Beads with the T3 Three Beads Populations in Ratios which Provide Beads Mixture with the Composition of:

Methimazole 10 mg

T3: 4 mcg IR beads, 3 mcg 8 hrs lag beads and 3 mcg 18 hrs lag time beads.

Fill the methimazole and the 3 T3 type beads into size 1 hard gelatin capsules

### Example 2

### Bimodal Modified Release FDC Tablets or Bead-Filed Capsules—Methimazole in SR Mode and T3 in Pulsatile Delivery Mode

Weigh 100 gr sugar beads (non-pareils—NP's), average size of 500 micron

Solubilize in 50:50 ethanol/water mixture about 2 gr Liothyronine, together with 0.1 gr PVP K30 and 0.02 gr NaCl

Spray the T3/PVP solution over the NP's, use Fluid bed equipped with Wurster column.

Weigh the IR T3 coated NP's and split into 3 subgroups:

### 1. Non-Coated IR Beads

Delayed Release Beads with Various Release Times for Pulsatile Delivery

### 2. Prepare Coating Solution Composed of Ethylcellulose 7 cp 80%, Triethylcitrate (TEC) 10% and PEG 6000 10%.

2a—Coat the Second IR T3 Coated Subgroup with 10% w/w of the Previous Composition, Enabling Delayed Release of the T3 after 8 Hrs Lag Time.
2b—Coat the Third IR T3 Coated Subgroup with 25% w/w of the Previous Composition, Enabling Delayed Release of the T3 after 18 Hrs Lag Time.

Weigh 100 gr sugar beads (NP's), average size of 500 microns

3. Solubilize in 50:50 Ethanol/Water Mixture about 20 gr Methimazole and 1 gr of PVP K30
4. Spray the Methimazole/PVP Solution Over the NP's, Use Fluid Bed Equipped with Wurster Column, Creating Methimazole IR Beads.
4a—Prepare Ethocel 20 cp/DBS (85/15 Ratios) Mixture and Solubilize in Ethanol
4a—Spray the Methimazole IR Beads with the Ethocel/DBS Mixture, Creating 15% w/w of Coating Layer, Converting the Methimazole IR Beads into 24 Hrs ER Beads
5. Mix the Methimazole ER Beads with the T3 Three Beads Populations in Ratios which Provide Beads Mixture with the Composition of:

Methimazole ER 10 mg

T3: 4 mcg IR beads, 3 mcg 8 hrs lag beads and 3 mcg 18 hrs lag time beads.

Fill the methimazole and the 3 T3 type beads into size 1 hard gelatin capsules

### Example 3

### Bimodal Modified Release FDC Tablets or Bead-Filed Capsules—Methimazole in IR/SR Mode and T3 in Pulsatile Delivery Mode

Weigh the three T3 beads types obtained as described in Examples 1 and 2 (IR, 8 hrs, 18 hrs) and mix with methimazole IR&ER beads prepared according to the previous samples.

Prepare hard gelatin capsules containing:

10 mcg of the T3 different beads and 5 mg methimazole IR beads together with 5 mg methimazole ER beads.

1. A bimodal modified release fixed-dose combination (FDC) composition comprising a first active agent which is triiodothyronine (T3) or a T3 functional analog and a second active agent which is at least one anti-thyroid agent.

2. The bimodal modified release FDC composition of claim 1, wherein the T3 functional analog is selected from an iodine derivative of thyronine, 3′,5′,3-triiodothyronine (Rt3), Rt3 derived hormone, 3′,3-diiodothyronine, 3′,5′-diiodothyronine, 5′,3-diiodothyronine, 3′-iodothyronine, 5′-iodothyronine, 3-iodothyronine, 3′-bromo-5′-iodo-3,5-di-iodothyronine, 3t-chloro-5′-iodo-3,5-diiodothyronine, isomer of 3,5,3′-triiodothyronine, triiodothyropropionic acid, L-isomer of tri-iodothyronine or an amide of iodothyronine.

3. The bimodal modified release FDC composition of claim 1, wherein the at least one anti-thyroid agent is at least one thioamide based drug selected from propylthiouracil (PTU), methimazole, carbimazole or combinations thereof.

4. The bimodal modified release FDC composition of claim 1, wherein the triiodothyronine (T3), or T3 functional analog is delivered in pulsatile release mode and the at least one anti-thyroid agent is delivered in immediate release (IR), sustained-release (SR) or combined IR/SR delivery modes.

5. The bimodal modified release FDC composition of claim 4, wherein comprising the amounts of triiodothyronine (T3) or T3 functional analog and of the at least one anti-thyroid agent necessary for once daily administration to a cancer patient in need thereof.

6. The bimodal modified release FDC composition of claim 4, wherein the T3 or T3 functional analog is delivered in pulsatile delivery mode in 2-6 pulses per 24 hrs., preferably 2-3 pulses per 24 hrs.

7. The bimodal modified release FDC composition of claim 4, wherein the at least one anti-thyroid agent is delivered in immediate-release delivery mode.

8. The bimodal modified release FDC composition of claim 4, wherein the at least one anti-thyroid agent is delivered in sustained-release delivery mode, and wherein at least 80% of the anti-thyroid agent is delivered over a 24 hrs. period.

9. The bimodal modified release FDC composition of claim 4, wherein the at least one anti-thyroid agent is delivered in a combined immediate release/sustained-release delivery mode, and wherein at least 80% of the anti-thyroid agent is delivered over a 24 hrs. period.

10. The bimodal modified release FDC composition of, claim 1, wherein formulated as a tablet or a capsule for oral administration.

11. The bimodal modified release FDC composition, claim 1, wherein formulated as an injectable composition for depot administration.

12. The bimodal modified release FDC composition of claim 5, wherein comprising from 15 μg to 90 μg triiodothyronine (T3) or a T3 functional analog thereof and from 10 mg to 1200 mg of at least one anti-thyroid agent.

13. The bimodal modified release FDC composition of claim 5, wherein comprising from 5 μg to 40 μg triiodothyronine (T3) or a T3 functional analog thereof and from 10 mg to 80 mg of methimazole, or from 100 mg to 1200 mg propylthiouracil (PTU) for daily administration.

14. The modified release FDC composition of claim 5, wherein comprising from 10 μg to 100 μg triiodothyronine (T3) or a T3 functional analog and from 5 mg to 100 mg of methimazole, or from 20 mg to 1000 mg propylthiouracil (PTU) for daily administration.

15. A method of treatment of cancer by administration of the bimodal modified release FDC composition of claim 1 to a cancer patient in need thereof.

16. The method of treatment of cancer of claim 15, wherein the cancer is a solid cancer selected from the group consisting of vascularized tumors, gonadal cancers, gastrointestinal, esophageal cancer, Barrett's esophagus cancer, oral cancer, parotid cancer, nasopharyngeal cancer, thyroid cancer, CNS cancers, urogenital cancers, skin cancers, bone cancers, connective tissue cancers and metastatic brain cancer, glioma and glioblastoma.

17. The method of treatment of cancer of claim 15, wherein the administration of the FDC composition is accompanied by anti-thyroid treatment with radioiodine and/or surgical removal of all or part of the thyroid gland.

18. A regimen of administration of the bimodal modified release FDC composition of claim 10 by administration to a cancer patient in need thereof of 1-4 tablets or capsules per day, preferably one tablet or capsule of the FDC composition per day.
